(19) United States
(12) Patent Application Publication
(10) Pub. No.: US 2015/0337267 A1
(43) Pub. Date: Nov. 26, 2015

(54) REPROGRAMMED STEM CELL

(71) Applicant: KYOTO UNIVERSITY, Kyoto-shi, Kyoto (JP)

(72) Inventors: Takashi TADA, Kyoto-shi, Kyoto (JP); Shogo NAGATA, Kyoto-shi, Kyoto (JP); Kunio HIRANO, Kyoto-shi, Kyoto (JP)

(21) Appl. No.: 14/759,027

(22) PCT Filed: Dec. 27, 2013
(86) PCT No.: PCT/JP2013/085352

§ 371 Date: Jul. 2, 2015

Related U.S. Application Data
(60) Provisional application No. 61/749,069, filed on Jan. 4, 2013.

(51) Int. Cl. C12N 5/074; C12N 5/0797

(52) U.S. Cl. CPC C12N 5/0696; C12N 5/0623; C12N 2510/02; C12N 2501/605; C12N 2501/60; C12N 2501/727

(57) ABSTRACT

The present invention provides a production method of a novel reprogrammed stem cell, including (1) introducing a reprogramming gene into a somatic cell and (2) selecting a cell wherein the exogenous reprogramming gene is completely free of expression suppression, and a novel reprogrammed stem cell produced by this method. The present invention further provides a production method of a pluripotent stem cell or a neural stem cell from the novel reprogrammed stem cell obtained by this method.

### TECHNICAL FIELD

The present invention relates to a self-renewable cultured cell, wherein an exogenous reprogramming gene has been introduced and the exogenous reprogramming gene is completely free of epigenetic expression suppression. The present invention also relates to a production method of a self-renewable cultured cell, comprising introducing a reprogramming gene into a somatic cell and selecting a cell wherein the exogenous reprogramming gene is completely free of expression suppression. Furthermore, the present invention relates to a production method of a pluripotent stem cell or neural stem cell from the above-mentioned cultured cells.

### BACKGROUND ART

Human ES cell and human iPS cell have been established, and various utilization methods have been considered, such as experimental materials of human embryology, application to regenerative medicine as a cell transplantation therapy, utilization for the development of a pharmaceutical product and the like.

However, when culturing human pluripotent stem cells such as human ES cell and human iPS cell, single dissociation results in apoptosis, unlike mouse pluripotent stem cells, and therefore, they need to be cultivated after colony formation. To clone a genetically-engineered cell, expansion culture from a single cell is necessary. In this event, cloning needs to be performed under special conditions such as use of Rho kinase inhibitor and the like.

### SUMMARY OF THE INVENTION

### Problems to be Solved by the Invention

The present invention aims to provide a novel reprogrammed stem cell different from an iPS cell. The present invention also aims to provide a production method of a pluripotent stem cell or neural stem cell from the novel reprogrammed stem cell.

### Means of Solving the Problems

The present inventors have found a self-renewal stem cell in the production process of iPS cell, and studied the stem cell. As a result, they have found that the cell can be easily cultured by single dissociation culture, and can be converted to a pluripotent stem cell by performing a high-density culture, and found for the first time that it is a useful stem cell, which resulted in the completion of the present invention.

Accordingly, the present invention encompasses the following.

[1] A self-renewable cultured cell, wherein an exogenous reprogramming gene has been introduced and the exogenous reprogramming gene is completely free of epigenetic expression suppression, wherein the aforementioned reprogramming gene is one or more genes selected from the group consisting of an Oct family gene, a Sox family gene, a Myc family gene and a Klf family gene.
[2] The cell according to [1], wherein the aforementioned reprogramming gene is an Oct family gene, a Sox family gene, a Myc family gene and a Klf family gene.
[3] The cell according to [1] or [2], wherein the aforementioned Oct family gene is Oct3/4, the aforementioned Sox family gene is Sox2, the aforementioned Myc family gene is c-Myc, and the aforementioned Klf family gene is Klf4.
[4] The cell according to any of [1] to [3], wherein the aforementioned reprogramming gene is incorporated into a chromosome.
[5] The cell according to any of [1] to [4], wherein endogenous Oct3/4 is not expressed, and NANOG, ZEB1 and ZEB2 are expressed.
[6] The cell according to any of [1] to [5], wherein expression of the exogenous reprogramming gene is suppressed by high-density culture.
[7] The cell according to any of [1] to [6], wherein expression of the endogenous Oct3/4 increases by high-density culture.
[8] The cell according to any of [1] to [7], wherein trimethylation of histone H3 lysine 9 increases by high-density culture.
[9] A production method of a self-renewable cultured cell, comprising
(1) introducing a reprogramming gene into a somatic cell and
(2) selecting a cell wherein the exogenous reprogramming gene is completely free of expression suppression,
wherein the aforementioned reprogramming gene is one or more genes selected from the group consisting of an Oct family gene, a Sox family gene, a Myc family gene and a Klf family gene.
[10] The method according to [9], wherein the aforementioned reprogramming gene is an Oct family gene, a Sox family gene, a Myc family gene and a Klf family gene.
[11] The method according to [9] or [10], wherein the aforementioned Oct family gene is Oct3/4, the aforementioned Sox family gene is Sox2, the aforementioned Myc family gene is c-Myc, and the aforementioned Klf family gene is Klf4.
[12] The method according to any of [9] to [11], wherein the aforementioned reprogramming gene is introduced by a retrovirus.
[13] The method according to any of [9] to [12], wherein the aforementioned cell does not express endogenous Oct3/4, but expresses NANOG, ZEB1 and ZEB2.
[14] A production method of a pluripotent stem cell, comprising high-density culture of the cultured cell described in any of [1] to [8].
[15] The method according to [14], wherein the aforementioned high-density culture is performed at a cell density of 1.5×10⁵ cells/cm² or more.
[16] The method according to [14] or [15], wherein the aforementioned pluripotent stem cell suppresses expression of an exogenous gene.
[17] The method according to any of [14] to [16], comprising using a medium added with an mTOR activator during the aforementioned high-density culture.
[18] The method according to [17], wherein the mTOR activator is VPA.
[19] A production method of a neural stem cell, comprising cultivating the cultured cell described in any of [1] to [8] in a medium added with a GSK3β inhibitor.
[20] The method according to [19], wherein the aforementioned GSK3β inhibitor is CHIR99021.
[21] The method according to [19] or [20], comprising further adding an MEK inhibitor to the aforementioned medium.
[22] The method according to [21], wherein the aforementioned MEK inhibitor is PD0325901.

### Effect of the Invention

Since the novel reprogrammed stem cell of the present invention permits single dissociation culture, genetic engineering can be performed easily. Furthermore, since the novel reprogrammed stem cell can be converted to a pluripotent stem cell by high-density culture, a pluripotent stem cell having a marker gene and the like, which is introduced by genetic engineering, can be obtained, which enables development of a therapeutic drug for diseases.

### BRIEF DESCRIPTION OF THE DRAWINGS

FIG. 1A shows a phase contrast microscopic image (top) and a fluorescence microscopic image for DsRed (bottom), of intermediately reprogrammed stem cells (iRS cells). FIG. 1B shows magnified images of a phase contrast microscopic image of intermediately reprogrammed stem cells.

FIG. 2A shows the results of hierarchical cluster analysis of the gene expression patterns of fibroblasts (TIG1), iRS cells, IPS cells and ES cells by microarray (left) and the plotted data of the ratio of the expression levels of each gene of TIG1 and iPS cells relative to that of iRS cell (right). FIG. 2B shows the results of expression analysis by PCR of each marker gene of TIG1, iRS cells, iPS cells and ES cells.

FIG. 3A shows phase contrast microscopic images (top) and fluorescence microscopic images for DsRed (bottom) when iRS cells were cultured at high density for 1 day, 3 days, 6 days and 10 days. FIG. 3B is a graph showing the expression levels of exogenous Oct4 and Sox2 after each culture period. FIG. 3C is a graph showing changes in the expression levels of endogenous Oct4, TDGF1 and ECAD after each culture period. FIG. 3D shows the results of hierarchical cluster analysis of each gene expression pattern by microarray after each culture period. FIG. 3E shows immunostained images for SSEA4 (left) and ECAD (right) using their antibodies, and fluorescence microscopic images for DsRed (bottom) after each culture period.

FIG. 4A shows changes in the rate of DsRed-negative cells when iRS cells were cultured at high density in a medium added with DMSO (control), sodium valproate (VPA), Rapamycin or VPA+Rapamycin. FIG. 4B shows the results of protein content of phosphorylated mTOR (p-mTOR) in iRS cells when DMSO (control), VPA or Rapamycin was added. CM is conditioned medium.

FIG. 5 shows immunostained images of iRS cells at 1 day, 3 days, and 6 days after high-density culture, when antibodies specific to the methylated or acetylated lysine residue of histone H3 and histone H4 were used.

FIG. 6A shows fluorescence microscopic images for DsRed (top) and immunostained images using an antibody to trimethylated histone H3 lysine 9 residue (H3K9) (bottom), of iRS cells at 1 day, 3 days, and 6 days after high-density culture. FIG. 6B shows fluorescence microscopic images for DsRed (top) and immunostained images using antibodies to trimethylated (middle) and acetylated (bottom) histone H3 lysine 27 residues (H3K27), of iRS cells at 1 day, 3 days, and 6 days after high-density culture. FIG. 6C shows fluorescence microscopic images for DsRed (top) and immunostained images using an antibody to dimethylated histone H3 lysine 36 residue (H3K36) (bottom), of iRS cells at 1 day, 3 days, and 6 days after high-density culture.

FIG. 7A shows a phase contrast microscopic image of neural stem cells (iPNSC) induced from iRS cells using a medium supplemented with a GSK3β inhibitor and a MEK inhibitor. FIG. 7B shows immunostained images of TUJ1-positive cells (left), O4-positive cells (middle) and GFAP-positive cells (right), derived from neural stem cells. FIG. 7C shows phase contrast microscopic images showing differentiation induction of neural stem cells from iRS cells under the conditions of no addition (left), addition of MEK inhibitor alone (middle) and addition of GSK3β inhibitor alone (right).

### DESCRIPTION OF EMBODIMENTS

The present invention is explained in detail in the following.

The present invention provides a production method of a self-renewable cultured cell, comprising (1) introducing a reprogramming gene into a somatic cell and (2) selecting, from the obtained cells, a cell wherein the exogenous reprogramming gene is completely free of expression suppression. Since the cultured cell obtained here has novel properties completely different from those of iPS cell, it is referred to as Intermediately Reprogrammed Stem (iRS) cell.

In the present invention, the reprogramming gene may be composed of a gene or non-coding RNA specifically expressed by ES cell, or a gene or non-coding RNA playing an important role for the maintenance of undifferentiation of ES cell. Examples of the reprogramming gene include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Toll, beta-catenin, Lin28b, Sall1, Sall4, Esrrb, Nr5a2, Tbx3, Glis1, Zscan4, PARP-1, Rex1, Cyclin D, Pin1, hTERT, SV40LT, UTF1, IRX6, GLIS1, PITX2, DMRTB1, family genes thereof and the like. Here, the family gene means a group of genes encoding proteins that share a domain, three-dimensional and functional unit, or a motif, smaller characteristic structure, or the like. Examples of the Oct family gene include Oct3/4, Oct1, Oct2, Oct5, Oct6 and the like, and examples of the Sox family gene include Sox2, Sox1, Sox3, Sox15, Sox17 and the like. Examples of the Klf family gene include Klf4, Klf2, Klf5 and the like, and examples of the Myc family gene include c-Myc, N-Myc, L-Myc and the like.

These reprogramming genes may be used alone or in combination. Examples of the combination of the reprogramming genes include combinations described in WO 2007/069666, WO 2008/118820, WO 2009/007852, WO 2009/032194, WO 2009/058413, WO 2009/057831, WO 2009/075119, WO 2009/079007, WO 2009/091659, WO 2009/101084, WO 2009/101407, WO 2009/102983, WO 2009/114949, WO 2009/117439, WO 2009/126250, WO 2009/126251, WO 2009/126655, WO 2009/157593, WO 2010/009015, WO 2010/033906, WO 2010/033920, WO 2010/042800, WO 2010/050626, WO 2010/056831, WO 2010/068955, WO 2010/098419, WO 2010/102267, WO 2010/111409, WO 2010/111422, WO 2010/115050, WO 2010/124290, WO 2010/147395, WO 2010/147612, WO 2012/158561, WO 2012/112458, WO 2012/096552, WO 2012/060473, WO 2012/057052, Huangfu D, et al. (2008), Nat. Biotechnol., 26: 795-797, Shi Y, et al. (2008), Cell Stem Cell, 2: 525-528, Eminli S, et al. (2008), Stem Cells. 26: 2467-2474, Huangfu D, et al. (2008), Nat Biotechnol. 26: 1269-1275, Shi Y, et al. (2008), Cell Stem Cell, 3, 568-574, Zhao Y, et al. (2008), Cell Stem Cell, 3: 475-479, Marson A, (2008), Cell Stem Cell, 3, 132-135, Feng B, et al. (2009), Nat Cell Biol. 11: 197-203, R. L. Judson et al., (2009), Nat. Biotech., 27: 459-461, Lyssiotis C A, et al. (2009), Proc Natl Acad Sci USA. 106: 8912-8917, Kim J B, et al. (2009), Nature. 461: 649-643, Ichida J K, et al. (2009), Cell Stem Cell. 5: 491-503, Heng J C, et al. (2010), Cell Stem Cell. 6: 167-74, Han J, et al. (2010), Nature. 463: 1096-100, Mali P, et al. (2010), Stem Cells. 28: 713-720, Maekawa M, et al. (2011), Nature. 474: 225-9. In the present invention, a preferable combination of the reprogramming genes is a combination of Oct family gene, Sox family gene, Klf family gene and Myc family gene, and more preferable combination of the reprogramming genes is Oct3/4, Sox2, Klf4 and c-Myc.

The present invention may also use, as a non-coding RNA for the reprogramming gene, miRNA, siRNA, shRNA and the like. Examples of the miRNA include hsa-mir-302a, hsa-miR-302b, hsa-miR-302c, hsa-miR-302d, hsa-miR-372, hsa-miR-373, hsa-miR-17, hsa-miR-20a, hsa-miR-20b, hsa-miR-93, hsa-mir-106a, hsa-mir-106b and has-mir-520d. These miRNAs can be confirmed in the websites such as miRBase (www.mirbase.org/) and the like, and reference may also be made to WO 2009/058413, WO 2009/075119, WO 2009/091659, WO 2010/115050, WO 2011/060100, WO 2011/102444, WO/2011/133288 and WO 2012/008302. Examples of the siRNA or shRNA include siRNA or shRNA against p53, siRNA or shRNA against antisense RNA of Oct3/4, Sox2 or Klf4, and siRNA or shRNA against p21. These siRNAs or shRNAs can be obtained by referring to WO 2009/157593, WO 2010/135329 and WO/2012/064090.

In the present invention, when a certain endogenous reprogramming gene is expressed in a somatic cell, into which a reprogramming gene is to be introduced, the reprogramming gene does not need to be introduced.

In the present invention, a reprogramming gene can be introduced into a somatic cell by the method of, for example, vector of virus, plasmid, artificial chromosome and the like, lipofection, liposome, microinjection and the like. Examples of the virus vector include retrovirus vector, lentivirus vector (hereinafter, Cell, 126, pp. 663-676, 2006; Cell, 131, pp. 861-872, 2007; Science, 318, pp. 1917-1920, 2007), adenovirus vector (Science, 322, 945-949, 2008), adeno-associated virus vector, vector of Hemagglutinating Virus of Japan (WO 2010/008054) and the like. Examples of the artificial chromosome vector include human artificial chromosome (HAC), yeast artificial chromosome (YAC), bacterial artificial chromosome (BAC, PAC) and the like. As the plasmid, plasmids for mammalian cells can be used (Science, 322: 949-953, 2008). The vector can contain regulatory sequences of promoter, enhancer, ribosome binding sequence, terminator, polyadenylation site and the like so that a reprogramming gene can be expressed and further, where necessary, a selection marker sequence of a drug resistance gene (e.g., kanamycin resistance gene, ampicillin resistance gene, puromycin resistance gene and the like), thymidine kinase gene, diphtheria toxin gene and the like, a reporter gene sequence of green fluorescent protein (GFP), β glucuronidase (GUS), FLAG and the like, and the like. Moreover, the above-mentioned vector may have an LoxP sequence before and after thereof to simultaneously cut out a gene encoding a reprogramming factor or a gene encoding a reprogramming factor bound to the promoter, after introduction into a somatic cell. In another preferred mode of embodiment, a method can be used wherein the transgene is integrated into chromosome using a transposon, thereafter a transposase is allowed to act on the cell using a plasmid vector or adenoviral vector so as to completely eliminate the transgene from the chromosome. As examples of preferable transposons, piggyBac, a transposon derived from a lepidopterous insect, and the like can be mentioned (Kaji, K. et al., (2009), Nature, 458: 771-775, Woltjen et al., (2009), Nature, 458: 766-770, WO 2010/012077). Furthermore, the vector may contain a sequence relating to the origin and replication of lymphotrophic herpes virus, BK virus and bovine papilloma virus, so that the gene is replicable even without integration into a chromosome and present episomally. For example, EBNA-1 and oriP or Large T and SV40ori sequence may be contained (WO 2009/115295, WO 2009/157201 and WO 2009/149233). In addition, for simultaneous introduction of plural reprogramming genes, an expression vector showing polycistronic expression may also be used. For polycistronic expression, the sequences encoding a gene may be linked by an IRES or foot-and-mouth disease virus (FMDV) 2A coding region (Science, 322: 949-953, 2008, WO 2009/092042 and WO 2009/152529).

In the present invention, moreover, a reprogramming gene may be introduced in the form of RNA or a protein. In the case of the form of a protein, for example, it may be fused with lipofection, or cell penetrating peptide (e.g., TAT derived from HIV and polyarginine) and contacted with the cell, or may be introduced into a somatic cell by techniques such as lipofection, microinjection and the like. When it is in the form of RNA, RNA incorporating 5-methylcytidine and pseudouridine (TriLink Biotechnologies) may be used to suppress degradation (Warren L, (2010) Cell Stem Cell. 7: 618-630). For introduction, techniques such as lipofection, microinjection and the like may be used. In the present invention, since a somatic cell incorporating a reprogramming gene preferably keeps expressing the gene, when it is introduced in the form of RNA or a protein, for example, the reprogramming gene needs to be introduced every other day, 2 days, 3 days, 4 days, 5 days, 6 days or 7 days.

In the present invention, it is preferable that the introduced reprogramming gene be incorporated into chromosome, and therefore, it is preferably introduced using a vector having retrovirus, lentivirus, or piggyBac.

In the present invention, culture is preferably continued after introduction of the gene into the somatic cell, so that the reprogramming gene will be stably expressed. For culture, it may be carried out in any medium in a coating-treated culture vessel, or carried out on a feeder cell. Examples of the feeder cell include mouse fibroblast (MEF), STO cell and the like. Examples of the coating agent include Matrigel (BD), collagen, gelatin, laminin, heparan sulfate proteoglycan, entactin, and a combination thereof. Preferred is a method including introducing a reprogramming gene into a somatic cell, culturing the cell on a feeder cell, transferring the cell into a Matrigel-coated culture vessel, and continuing the culture.

The culture broth used for the culture in the present invention can be prepared using, as a basal medium, a medium used for culturing animal cells. Examples of the basal medium include MEM, 199 medium, Eagle's Minimum Essential Medium (EMEM), αMEM, Dulbecco's modified Eagle's Medium (DMEM), Ham's F12 medium, RPMI 1640 medium, Fischer's medium, Neurobasal Medium (Life Technologies), a mixed medium of these and the like. The medium may contain serum, or may be serum-free. Where necessary, the medium may contain, for example, one or more serum replacements such as albumin, transferrin, Knockout Serum Replacement (KSR) (serum replacement of FBS for ES cell culture), N2 supplement (Invitrogen), B27 supplement (Invitrogen), fatty acid, insulin, collagen precursor, trace element, 2-mercaptoethanol, 3′-thiolglycerol and the like, and can also contain one or more substances such as lipid, amino acid, L-glutamine, Glutamax (Invitrogen), non-essential amino acid, vitamin, growth factor (bFGF etc.), low-molecular-weight compound, antibiotic, antioxidant, pyruvic acid, buffering agent, inorganic salts and the like. Preferable medium includes MEM containing 10% serum and a 1:1 mixture of DMEM and F12 containing KSR and bFGF. A more preferable medium is the culture supernatant (conditioned medium) of a medium in which MEF and the like were cultured.

As regards the culture conditions, while the culture temperature is not limited to the following, it is about 30-about 40° C., preferably about 37° C., and the culture is performed under the atmosphere of CO₂-containing air, where the CO₂ concentration is preferably about 2-about 5%.

While the culture period is not particularly limited, it is, for example, 10 days or more, 15 days or more, 20 days or more, 25 days or more, 30 days or more, 35 days or more, 40 days or more, or more number of days. Preferred is 20 days or more.

After the culture, iRS cell can be selected from the obtained cell group. The selection of the iRS cell can be performed based on the absence of suppression of the expression of the introduced reprogramming gene. For example, when a marker gene is introduced together with the reprogramming gene, the expression of the marker gene can be confirmed. For confirmation of the expression of the marker gene, when the marker gene is a drug resistance gene, iRS cell can be selected by culturing in a culture medium (selection culture medium) containing a corresponding drug. When the marker gene is a fluorescent protein gene, iRS cell can be selected by observation with a fluorescence microscope, when it is a luminescent enzyme gene, iRS cell can be selected by adding a luminescent substrate, and when it is a chromogenic enzyme gene, iRS cell can be selected by adding a chromogenic substrate.

In the present invention, a somatic cell to be introduced with a reprogramming gene means any animal cell (preferably, cells of mammals inclusive of human) excluding germ line cells and totipotent cells such as ovum, oocyte, ES cells and the like. While somatic cell is not particularly limited, it encompasses any of somatic cells of fetuses, somatic cells of neonates, and mature healthy or pathogenic somatic cells, and any of primary cultured cells, passage cells, and established lines of cells. Specific examples of the somatic cell include (1) tissue stem cells (somatic stem cells) such as neural stem cell, hematopoietic stem cell, mesenchymal stem cell, dental pulp stem cell and the like, (2) tissue progenitor cell, (3) differentiated cells such as lymphocyte, epithelial cell, endothelial cell, myocyte, fibroblast (skin cells etc.), hair cell, hepatocyte, gastric mucosal cell, enterocyte, splenocyte, pancreatic cell (pancreatic exocrine cell etc.), brain cell, lung cell, renal cell and adipocyte and the like, and the like.

### <iRS Cell>

The iRS cell obtained by the above-mentioned method is characterized in that it grows by self-renewal, and the expression of the introduced reprogramming gene is not suppressed even after successive passage cultures. Furthermore, iRS cell is also characterized by the presence or absence of the expression of a specific marker gene. For example, a cell showing no expression or a significantly low expression of a marker gene selected from endogenous Oct3/4, Klf4, c-Myc, TDGF1, Rex1, E-cadherin (ECAD) and EPCAM as compared to pluripotent stem cells such as ES cell, iPS cell and the like, and showing an expression of a marker gene selected from Nanog, ZEB1 and ZEB2, preferably, a cell showing a significantly higher expression of Nanog than somatic cells and a significantly higher expression of ZEB1 and/or ZEB2 than pluripotent stem cells, is taken as an iRS cell. More preferably, iRS cell is a cell free of expression of endogenous Oct3/4 and expressing Nanog, ZEB1 and ZEB2, more preferably, a cell expressing Nanog equivalently to pluripotent stem cells and expressing ZEB1 and ZEB2 equivalently to somatic cells. Other than these, a cell showing no or a significantly low modification of monomethylation (H3K4me1), dimethylation (H3K4me2) and trimethylation (H3K4me3) of histone H3 lysine 4 (H3K4), monomethylation (H3K9me1), dimethylation (H3K9me2), trimethylation (H3K9me3) and acetylation (H3K9Ac) of histone H3 lysine 9 (H3K9), acetylation (H3K14Ac) of histone H3 lysine 14 (H3K14), monomethylation (H3K27me1), dimethylation (H3K27me2) and trimethylation (H3K27me3) of histone H3 lysine 27 (H3K27), monomethylation (H3K36me1), dimethylation (H3K36me2) and trimethylation (H3K36me3) of histone H3 lysine 36 (H3K36), acetylation (H4K8Ac) of histone H4 lysine 8 (H4K8) and monomethylation (H4K20me1) of histone H4 lysine 20 (H4K20), as compared to pluripotent stem cells can be used as an iRS cell.

In the present invention, the expression of a marker gene may be verified by measuring the RNA level by a nucleic acid amplification test, or may be verified by determining the amount of the translation product by using a specific antibody.

iRS cell can be amplified by passage culture. For passaging, the cells can be dissociated to allow for dissociation into single cells. Examples of the method for cell dissociation include a method including mechanical dissociation, and a dissociation method using a dissociation solution having a protease activity and collagenase activity (e.g., trypsin solution, Accutase™, Accumax™ and the like) or a dissociation solution having collagenase activity alone.

For passage culture of iRS cell in the present invention, the culture may be performed in any medium in a coating-treated culture vessel. Examples of the coating agent include Matrigel (BD), collagen, gelatin, laminin, heparan sulfate proteoglycan, entactin, and a combination of these. Preferred is a method including introducing a reprogrammed gene into a somatic cell, culturing the cell on a feeder cell, transferring the cell to a Matrigel-coated culture vessel, and continuing the culture.

The culture broth used for passage culture of iRS cell in the present invention can be prepared using, as a basal medium, a medium used for culture of animal cells. Examples of the basal medium include MEM, 199 medium, Eagle's Minimum Essential Medium (EMEM), αMEM, Dulbecco's modified Eagle's Medium (DMEM), Ham's F12 medium, RPMI 1640 medium, Fischer's medium, Neurobasal Medium (Life Technologies), a mixed medium of these and the like. The medium may contain serum, or may be serum-free. Where necessary, the medium may contain, for example, To one or more serum replacements such as albumin, transferrin, Knockout Serum Replacement (KSR) (serum replacement of FBS for ES cell culture), N2 supplement (Invitrogen), B27 supplement (Invitrogen), fatty acid, insulin, collagen precursor, trace element, 2-mercaptoethanol, 3′-thiolglycerol and the like, and can also contain one or more substances such as lipid, amino acid, L-glutamine, Glutamax (Invitrogen), non-essential amino acid, vitamin, growth factor (bFGF etc.), low-molecular-weight compound, antibiotic, antioxidant, pyruvic acid, buffering agent, inorganic salts and the like. Preferable medium is a 1:1 mixture of DMEM and F12 containing bFGF and KSR. A more preferable medium is the culture supernatant (conditioned medium) of a medium in which MEF and the like were cultured.

As regards the culture conditions, while the culture temperature is not limited to the following, it is about 30-about 40° C., preferably about 37° C., and the culture is performed under the atmosphere of CO₂-containing air, where the CO₂ concentration is preferably about 2-about 5%.

The passage period is, for example, preferably within 2 days, within 3 days, within 4 days, or within 5 days, since adhesion of the cells influences self-renewal of iRS cells. Preferred is 3 days.

### <Method of Conversion to Pluripotent Stem Cell>

The iRS cell obtained by the above-mentioned method can be converted to a pluripotent stem cell by culturing at high-density. Here, the pluripotent stem cell is a stem cell having pluripotency permitting differentiation into any cell in living organisms, and also having self-proliferative capacity.

The high-density culture for conversion of an iRS cell to a pluripotent stem cell only requires cells to be in contact with each other. While the density is not particularly limited, it is, for example, not less than 5×10⁴ cells/cm², not less than 1×10⁵ cells/cm², not less than 1.5×10⁵ cells/cm², not less than 2×10⁵ cells/cm², not less than 2.5×10⁵ cells/cm², not less than 3×10⁵ cells/cm² or not less than 3.5×10⁵ cells/cm².

In the present invention, for high-density culture of iRS cell, the culture may be performed in any medium in a coating-treated culture vessel. Examples of the coating agent include Matrigel (BD), collagen, gelatin, laminin, heparan sulfate proteoglycan, entactin, and a combination of these. Preferred is a method including introducing a reprogrammed gene into a somatic cell, culturing the cell on a feeder cell, transferring the cell to a Matrigel-coated culture vessel, and continuing the culture.

The culture broth used for high-density culture of iRS cell in the present invention can be prepared using, as a basal medium, a medium used for culture of animal cells. Examples of the basal medium include MEM, 199 medium, Eagle's Minimum Essential Medium (EMEM), αMEM, Dulbecco's modified Eagle's Medium (DMEM), Ham's F12 medium, RPMI 1640 medium, Fischer's medium, Neurobasal Medium (Life Technologies), a mixed medium of these and the like. The medium may contain serum, or may be serum-free. Where necessary, the medium may contain, for example, one or more serum replacements such as albumin, transferrin, Knockout Serum Replacement (KSR) (serum replacement of FBS for ES cell culture), N2 supplement (Invitrogen), B27 supplement (Invitrogen), fatty acid, insulin, collagen precursor, trace element, 2-mercaptoethanol, 3′-thiolglycerol and the like, and can also contain one or more substances such as lipid, amino acid, L-glutamine, Glutamax (Invitrogen), non-essential amino acid, vitamin, growth factor (bFGF etc.), low-molecular-weight compound, antibiotic, antioxidant, pyruvic acid, buffering agent, inorganic salts and the like. Preferable medium is a 1:1 mixture of DMEM and F12 containing bFGF and KSR. A more preferable medium is the culture supernatant (conditioned medium) of a medium in which MEF and the like were cultured.

Examples of the low-molecular-weight compound to be added include mTOR activators. Examples of the mTOR activator include mTOR activator and sodium valproate (VPA) described in WO 2006/027545; Foster, D. A., Cancer Res, 67(1): 1-4 (2007); and Tee et al., J. Biol. Chem. 278: 37288-96 (2003). A preferable mTOR activator in the present invention is VPA.

As regards the culture conditions, while the culture temperature is not limited to the following, it is about 30-about 40° C., preferably about 37° C., and the culture is performed under the atmosphere of CO₂-containing air, where the CO₂ concentration is preferably about 2-about 5%.

The high-density culture in the present invention is desirably performed at least for 6 days. Examples thereof include 6 days, 7 days, 8 days, 9 days and 10 days.

### <Method of Conversion to Neural Stem Cell>

The iRS cell obtained by the above-mentioned method can be converted to a neural stem cell by culturing in a medium added with a GSK3β inhibitor.

The neural stem cell in the present invention is a stem cell having an ability to supply cells to be differentiated into a neuron and a glial cell, and can be identified using an expression marker for primitive neuroectoderm or neural stem cell, such as neural cell adhesion molecule (NCAM), polysialylated NCAM, A2B5 (expressed in embryonic or neonatal nerve cells), intermediate filament proteins (nestin, vimentin and the like), transcription factor Pax-6 and the like, dopamine neuron markers (tyrosine hydroxylase (TH) and the like), neural markers (TuJ1 and the like) and the like.

The culture broth used for cultivating neural stem cell from iRS cell can be prepared using, as a basal medium, a medium used for culture of animal cells. Examples of the basal medium include IMDM, 199 medium, Eagle's Minimum Essential Medium (EMEM), αMEM, Dulbecco's modified Eagle's Medium (DMEM), Ham's F12 medium, RPMI 1640 medium, Fischer's medium, Neurobasal Medium (Life Technologies), a mixed medium of these and the like. Preferred is Neurobasal Medium. The medium may contain serum, or may be serum-free. Where necessary, the medium may contain, for example, one or more serum replacements such as albumin, transferrin, Knockout Serum Replacement (KSR) (serum replacement of FBS for ES cell culture), N2 supplement (Invitrogen), B27 supplement (Invitrogen), fatty acid, insulin, collagen precursor, trace element, 2-mercaptoethanol, 3′-thiolglycerol and the like, and can also contain one or more substances such as lipid, amino acid, L-glutamine, Glutamax (Invitrogen), non-essential amino acid, vitamin, growth factor, low-molecular-weight compound, antibiotic, antioxidant, pyruvic acid, buffering agent, inorganic salts and the like. Preferable medium is a 1:1 mixture of DMEM and F12 containing MEK inhibitor, GSK-3β inhibitor, N2 supplement, B27 supplement.

The GSK-3β inhibitor in the present invention is defined as a substance that inhibits kinase activity of GSK-3β protein (e.g., phosphorylation capacity against β catenin), and many are already known. Examples thereof include BIO, which is an indirubin derivative (alias, GSK-3β inhibitor IX; 6-bromo indirubin 3′-oxime), SB216763 which is a maleimide derivative (3-(2,4-dichlorophenyl)-4-(1-methyl-1H-indol-3-yl)-1H-pyrrole-2,5-dione), GSK-3β inhibitor VII which is a phenyl α-bromomethyl ketone compound (4-dibromoacetophenone), L803-mts which is a cell membrane-permeable type-phosphorylated peptide (alias, GSK-3β peptide inhibitor; Myr-N-GKEAPPAPPQSpP-NH₂) and CHIR99021 having high selectivity (6-[2-[4-(2,4-Dichlorophenyl)-5-(4-methyl-1H-imidazol-2-yl)pyrimidin-2-ylamino]ethylamino]pyridine-3-carbonitrile). These compounds are commercially available from, for example, Calbiochem, Biomol and the like, and can be easily utilized. They may be obtained from other sources, or may be directly produced.

The GSK-3β inhibitor used in the present invention can preferably be CHIR99021.

The concentration of CHIR99021 in a medium is, though not limited to, for example, 1 nM, 10 nM, 50 nM, 100 nM, 500 nM, 750 nM, 1 μM, 2 μM, 3 μM, 4 μM, 5 μM, 6 μM, 7 μM, 8 μM, 9 μM, 10 μM, 15 μM, 20 μM, 25 μM, 30 μM, 40 μM or 50 μM, preferably 3 μM.

The MEK inhibitor in the present invention is a drug having an action to inhibit movements of MEK (block cell proliferation signaling). MEK is a phosphorylating enzyme present in a cell proliferation signaling pathway (MAP kinase pathway) where a cell growth factor binds to a cell receptor, and the signal therefrom reaches nucleus. Examples of the MEK inhibitor include PD184352, PD98059, U0126, SL327, PD0325901 and the like.

The MEK inhibitor used in the present invention can preferably be PD0325901.

The concentration of PD0325901 in a medium is, though not limited to, for example, 1 nM, 10 nM, 50 nM, 100 nM, 500 nM, 750 nM, 1 μM, 2 μM, 3 μM, 4 μM, 5 μM, 6 μM, 7 μM, 8 μM, 9 μM, 10 μM, 15 μM, 20 μM, 25 μM, 30 μM, 40 μM or 50 μM, preferably 500 nM.

As regards the culture conditions, while the culture temperature is not limited to the following, it is about 30-about 40° C., preferably about 37° C., and the culture is performed under the atmosphere of CO₂-containing air, where the CO₂ concentration is preferably about 2-about 5%.

The high-density culture in the present invention is desirably performed at least for 6 days. Examples thereof include 6 days, 7 days, 8 days, 9 days and 10 days.

### <Kit for Production of iRS Cell>

The present invention provides a kit for production of iRS cells. The kit may contain the aforementioned reprogramming gene, transgene reagent, compound, culture medium, dissociation solution and coating agent for a culture vessel. This kit may further contain a protocol or instructions describing the step of differentiation induction.

### EXAMPLES

### Example 1

### Production of Intermediately Reprogrammed Stem (iRS) Cell

OCT3/4, SOX2, KLF4, c-MYC (Takahashi K, et al, Cell. 131, 861-872, 2007) and DsRed (Okita K, et al, Nature. 448, 313-317, 2007) were introduced into human fibroblasts TIG1 (Ohashi, M, et al, Exp. Gerontol., 15, 539-549, 1980) using retrovirus. Then, TIG1 (1×10⁵ cells) incorporating the gene was plated in a 3 cm-dish. MEF (4.0×10⁵ cells) was used as a feeder cell, and MEM containing 10% FBS was used as the medium. After 4 days of culture, the cells were detached with 0.25% trypsin/EDTA solution, and the transfected TIG1 (5×10⁴ cells) was plated in a 10 cm dish. In this case, MEF (2.5×10⁶ cells) was used as a feeder cell, and MEM containing 10% FBS was used as the medium.

The next day, the medium was exchanged with a MEF-conditioned medium obtained by culturing MEF in a human ES cell medium (DMEM/F12 containing KSR and bFGF) for 24 hr.

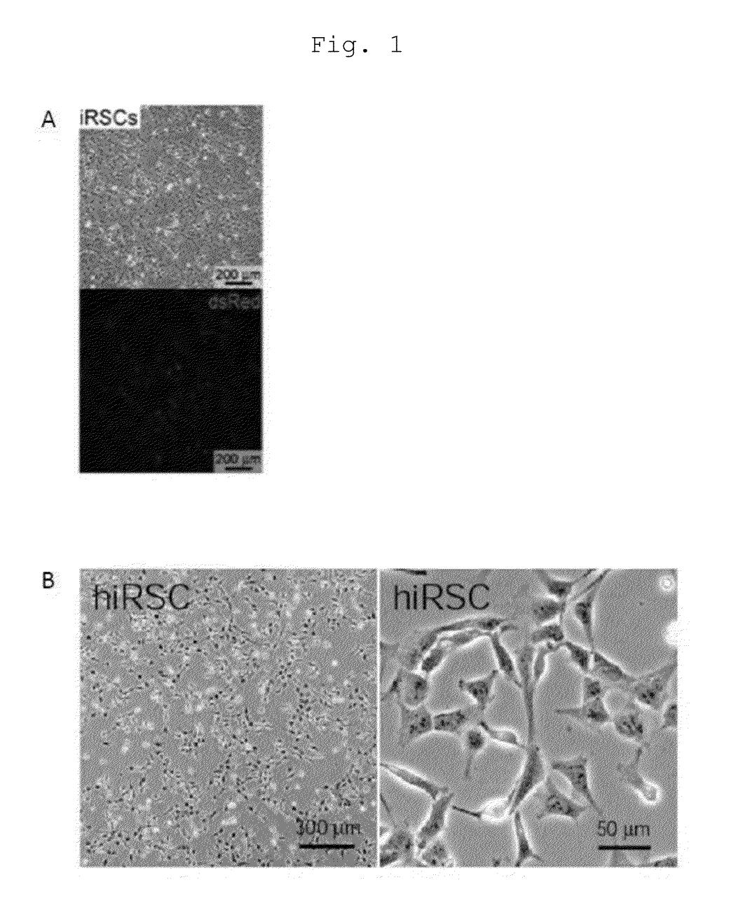

After 15-25 days from the medium exchange, colonies expressing DsRed from among the obtained colonies were transferred, by one colony, into a 1 cm well coated with 2% Matrigel (BD), and cultured in the MEF-conditioned medium. 5 days later, iRS cells expressing DsRed were obtained (FIG. 1A or B).

### Example 2

### Culture of iRS Cell

iRS cells (2.5×10⁵ cells) obtained by the above-mentioned method were cultured in a MEF-conditioned medium in a 3 cm dish coated with Matrigel. Every 2 days, the cells were detached with 0.25% trypsin/EDTA solution and passaged. In this case, the medium was exchanged every day.

### <Expression of Marker Gene in iRS Cell>

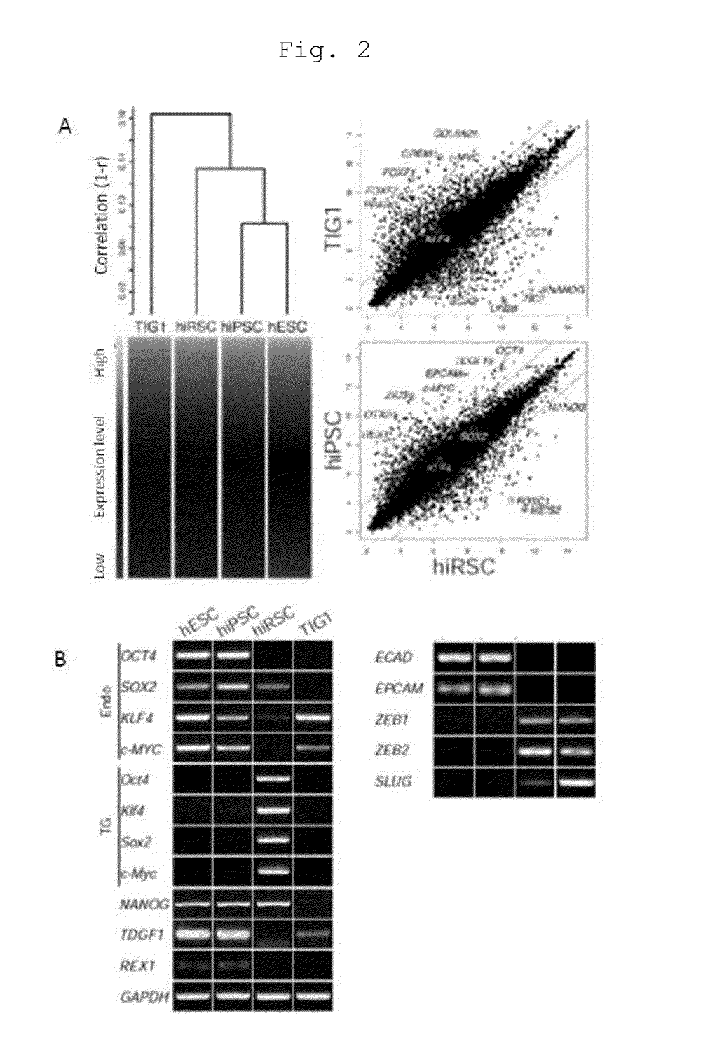

The iRS cells were subjected to gene analysis using microarray, and cluster analyzed together with human iPS cell (201B7 line, obtained from Kyoto University), human ES cell (obtained from Kyoto University) and TIG1. As a result, the gene expression profile of iRS cell was different from that of human iPS cell and ES cell, and further different from that of TIG1 (FIG. 2A). Moreover, using PCR, expression of OCT4, SOX2, KLF4 and c-MYC (exogenous and endogenous expressions were respectively detected for these), and NANOG, TDF1, REX1, E-cadherin (ECAD), EPCAM, ZEB1, ZEB2 and SLUG was detected. The results are shown in FIG. 2B.

### Example 3

### Production of Pluripotent Cell from iRS Cell

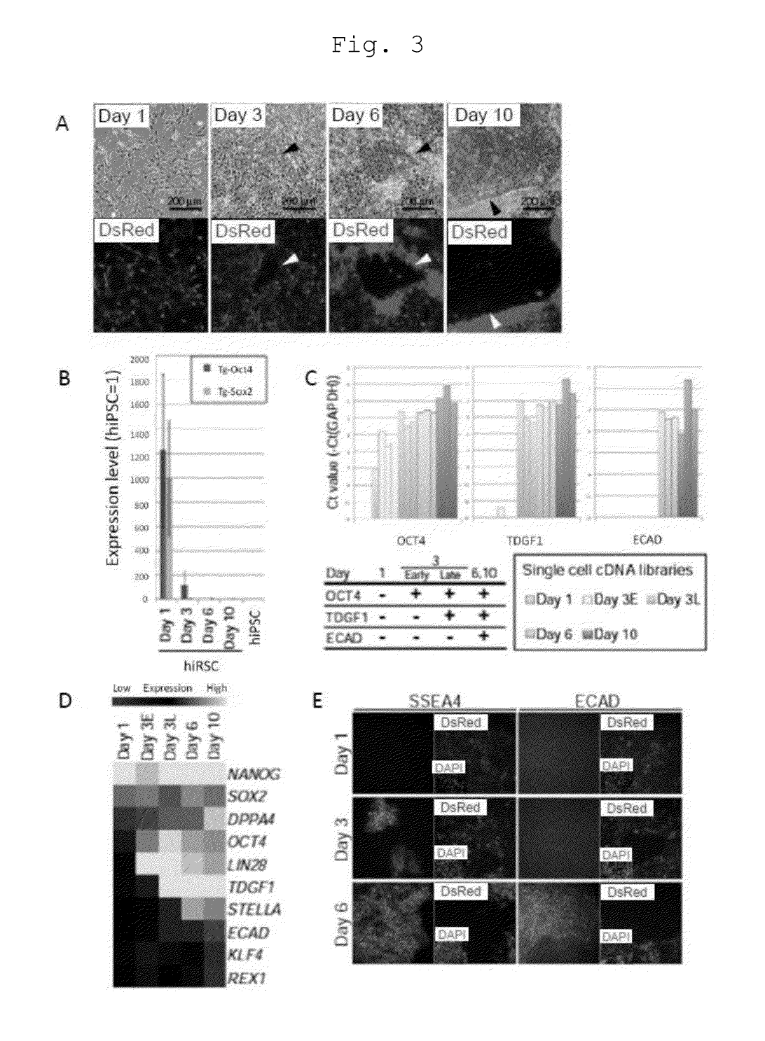

The iRS cells were plated at a high density (1×10⁶ cells) on a 3 cm dish coated with Matrigel, and culture was continued in a MEF-conditioned medium. From day 3, DsRed-negative cells were occasionally found, and DsRed-negative cell clump could be confirmed on day 6. On day 10, ES cell-like colony could be confirmed (FIG. 3A). The expression levels of exogenous OCT4 and SOX2 at that time were confirmed by quantitative PCR. Expression of exogenous gene disappeared on day 6 of high-density culture, and re-expression was void thereafter (FIG. 3B). Furthermore, the expression levels of the endogenous OCT4, TDGF1 and ECAD were confirmed by quantitative PCR. As a result, expression of these pluripotent marker genes was confirmed (FIG. 3C). Similarly, the mRNA expression levels of other pluripotent marker genes were confirmed on a heat map. As a result, mostly similar gene expression profiles were confirmed on day 6 and day 10 (FIG. 3D). Moreover, SSEA4 and ECAD, which are surface antigen markers showing pluripotency, were tested by an immunostaining method. As a result, both surface antigens could be recognized from day 6 (FIG. 3E). From the above, iRS cell could be converted into pluripotent cell by high-density culture.

### <Influence of Low-Molecular-Weight Compound on Conversion of iRS Cell to Pluripotent Cell>

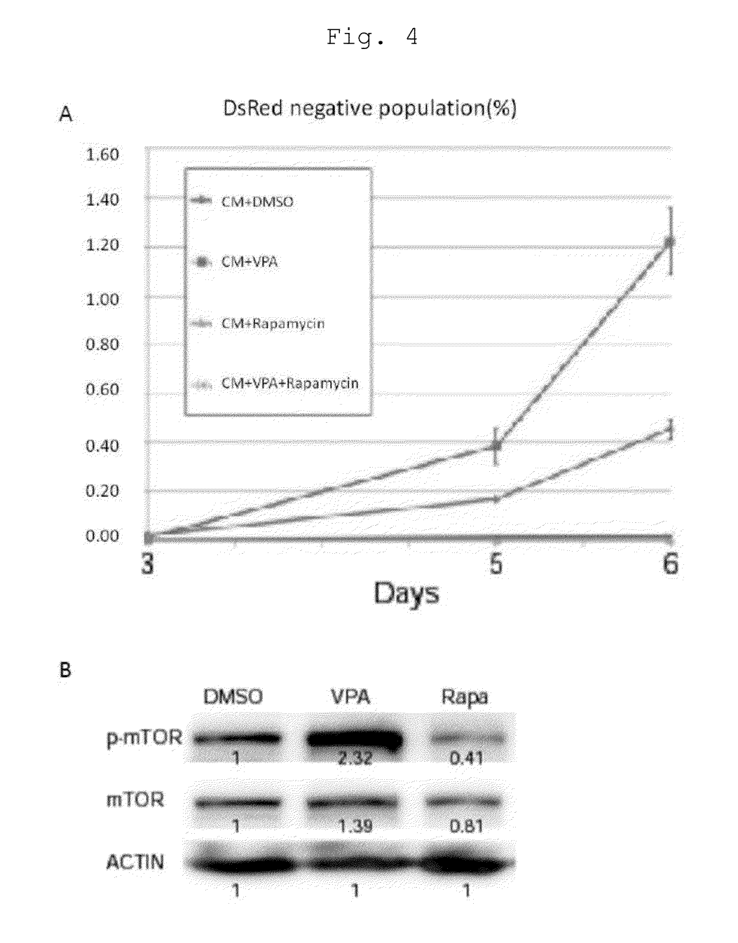

iRS cells were plated at a high density (1×10⁶ cells) on a 3 cm dish coated with Matrigel, 0.5 mM VPA was added to the MEF-conditioned medium and culture was continued. As a result, the conversion rate to DsRed-negative cells became strikingly high on day 6 after plating (FIG. 4A). On the other hand, when 20 nM rapamycin was added to the medium, DsRed-negative cell did not appear (FIG. 4A). Phosphorylation of mTOR then was measured. As a result, the amount of phosphorylated mTOR (p-mTOR) increased by the addition of VPA, and decreased by the addition of rapamycin (FIG. 4B). From the above, it was confirmed that mTOR signaling plays an important role in the conversion of iRS cell to pluripotent cell. Therefore, it was suggested that a low-molecular-weight compound (e.g., VPA) that enhances mTOR signaling is useful for conversion of iRS cell to pluripotent cell.

### <Histone Modification of iRS Cell>

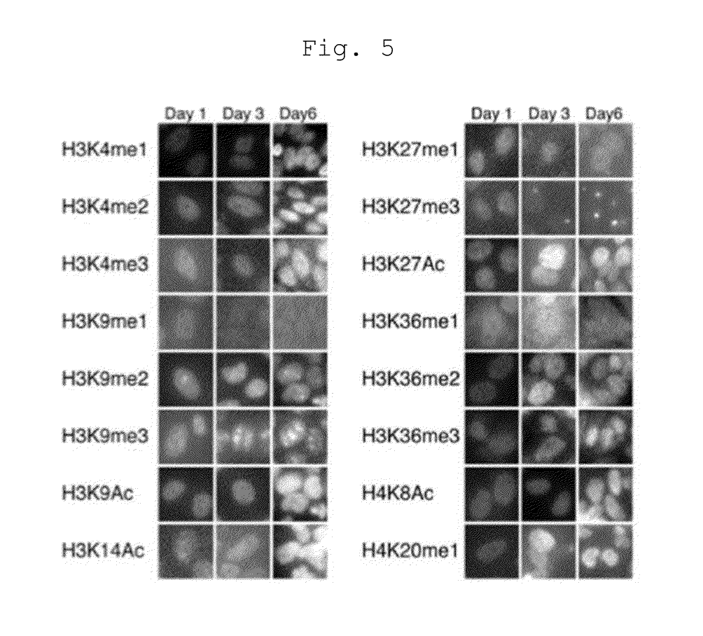

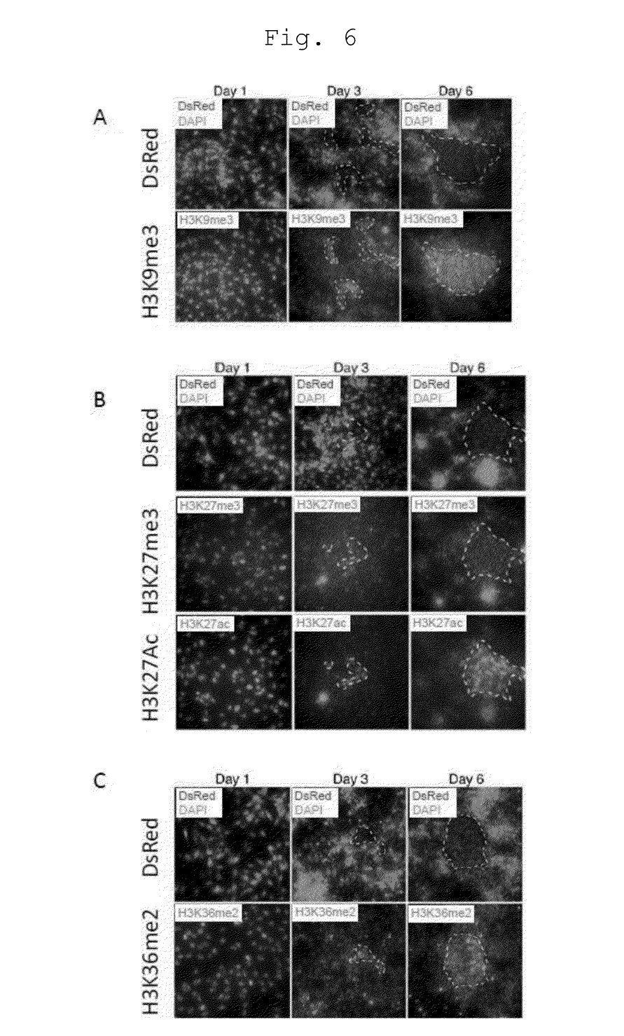

iRS cells were plated at a high density (1×10⁶ cells) on a 3 cm dish coated with Matrigel, and culture was continued in a MEF-conditioned medium. Methylation and acetylation of histones on day 1, day 3 and day 6 were measured by an immunostaining method. The results are shown in FIGS. 5 and 6. While the iRS cells on day 1 of high-density culture showed almost negative as for histone modification, it was confirmed that trimethylation of histone H3 lysine 9 (H3K9) (H3K9me3) (FIG. 6A), trimethylation of H3K27 (H3K27me3), acetylation of H3K27 (H3K27Ac) (FIG. 6B) and dimethylation of H3K36 (H3K36me2) (FIG. 6C) increased on day 3. Furthermore, it was confirmed that monomethylation of H3K4 (H3K4me1), dimethylation thereof (H3K4me2) and trimethylation thereof (H3K4me3), acetylation of H3K9 (H3K9Ac), acetylation of H3K14 (H3K14Ac), trimethylation of H3K36 (H3K36me3), K8 acetylation of histone H4 (H4) (H4K8Ac) and monomethylation of H4K20 (H4K20me1) were promoted on day 6, in addition to the above-mentioned modifications (FIG. 5). From the above, it was confirmed that high-density culture of iRS cell changes histone modification to confer pluripotency.

### Example 4

### Production of Neural Stem Cell from iRS Cell

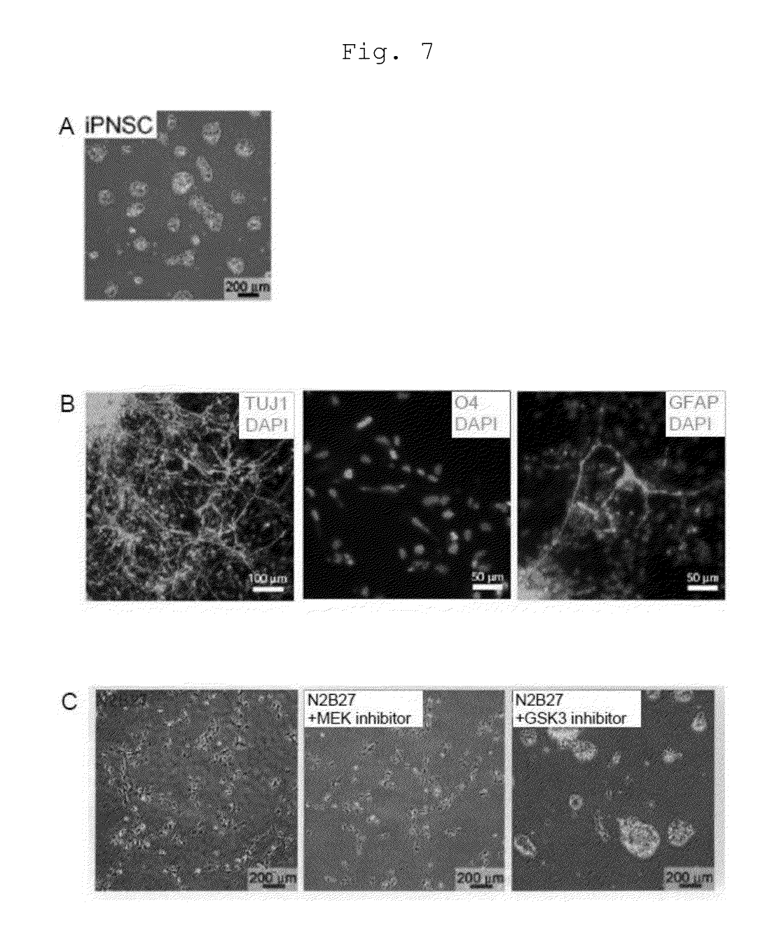

iRS cells were plated at a high density (1×10⁶ cells) on a 3 cm dish coated with Matrigel, and the medium was exchanged with DMEM/F12 containing N2 and B27 (N2B27) added with 3 μM CHIR99021 (GSK3β inhibitor) and 0.5 μM PD0325901 (MEK inhibitor), and the cells were cultured for 6 days. By colony pick up, neural stem cell-like cells appeared (FIG. 7A). The neural stem cell-like cells were continuously cultured, and stained with antibodies to TUJ1 (neuron marker gene), 04 (oligodendrocyte marker gene) and GFAP (astrocyte marker gene) by immunostaining. As a result, cells positive to these marker genes were confirmed. Therefore, it was confirmed that the neural stem cell-like cells were neural stem cells having differentiation ability into each nerve system cell (FIG. 7B). That is, it was confirmed that culture of iRS cells in a medium added with GSK3β inhibitor and MEK inhibitor enables induction into neural stem cells. Furthermore, similar results were confirmed even with the GSK3β inhibitor alone (FIG. 7C). From the above, it was confirmed that culture of iRS cells in a medium added with at least a GSK3β inhibitor enables induction of neural stem cells.

### INDUSTRIAL APPLICABILITY

The novel reprogrammed stem cell of the present invention is useful as a research tool for the development of a therapeutic drug for diseases, and the like.

This application is based on U.S. provisional patent application No. 61/749,069, the contents of which are encompassed in full herein.

1. A self-renewable cultured cell, wherein exogenous reprogramming genes have been introduced and the exogenous reprogramming genes are completely free of epigenetic expression suppression, wherein said reprogramming genes are an Oct family gene, a Sox family gene, a Myc family gene and a Klf family gene, wherein said reprogramming genes are incorporated into a chromosome, and wherein endogenous Oct3/4 is not expressed, and NANoG, ZEB1 and ZEB2 are expressed.

2. (canceled)

3. The cell according to claim 1, wherein said Oct family gene is Oct3/4, said Sox family gene is Sox2, said Myc family gene is c-Myc, and said Klf family gene is Klf4.

4.-5. (canceled)

6. The cell according to claim 1, wherein expression of the exogenous reprogramming genes are suppressed by high-density culture.

7. The cell according to claim 1, wherein expression of the endogenous Oct3/4 increases by high-density culture.

8. The cell according to claim 1, wherein trimethylation of histone H3 lysine 9 increases by high-density culture.

9. A method of producing a self-renewable cultured cell, comprising
(1) introducing a reprogramming gene into a somatic cell and
(2) selecting a cell wherein the exogenous reprogramming gene is completely free of expression suppression,
wherein said reprogramming gene is one or more genes selected from the group consisting of an Oct family gene, a Sox family gene, a Myc family gene and a Klf family gene.

10. The method according to claim 9, wherein said reprogramming gene is an Oct family gene, a Sox family gene, a Myc family gene and a Klf family gene.

11. The method according to claim 9, wherein said Oct family gene is Oct3/4, said Sox family gene is Sox2, said Myc family gene is c-Myc, and said Klf family gene is Klf4.

12. The method according to claim 9, wherein said reprogramming gene is introduced by a retrovirus.

13. The method according to claim 9, wherein said cell does not express endogenous Oct3/4, but expresses NANOG, ZEB1 and ZEB2.

14. A method of producing a pluripotent stem cell, comprising high-density culture of the cultured cell according to claim 1, wherein said pluripotent stem cell suppresses expression of an exogenous gene.

15. The method according to claim 14, wherein said high-density culture is performed at a cell density of 1.5×10⁵ cells/cm² or more.

16. (canceled)

17. The method according to claim 14, comprising using a medium added with an mTOR activator during said high-density culture.

18. The method according to claim 17, wherein the mTOR activator is VPA.

19. A method of producing a neural stem cell, comprising cultivating the cultured cell according to claim 1 in a medium added with a GSK3β inhibitor.

20. The method according to claim 19, wherein said GSK3β inhibitor is CHIR99021.

21. The method according to claim 19, comprising further adding an MEK inhibitor to said medium.

22. The method according to claim 21, wherein said MEK inhibitor is PD0325901.
